(19) United States
(12) Patent Application Publication
(10) Pub. No.: US 2023/0242863 A1
(43) Pub. Date: Aug. 3, 2023

(54) PIEZOELECTRIC MEMBRANE-MICROELECTRODE ARRAY

(71) Applicant: NMI Naturwissenschaftliches und Medizinisches Institut an der Universitaet Tuebingen, Reutlingen (DE)

(72) Inventors: Maximilian BECKER, Tuebingen (DE); Claus BURKHARDT, Tuebingen (DE)

(21) Appl. No.: 18/133,100

(22) Filed: Apr. 11, 2023

Related U.S. Application Data
(63) Continuation of application No. PCT/EP2021/077671, filed on Oct. 7, 2021.

(30) Foreign Application Priority Data
Oct. 12, 2020 (DE) 10 2020 126 759.2

(51) Int. Cl. C12M 1/34; C12M 1/42; C12M 3/06; C12M 1/12

(52) U.S. Cl. CPC C12M 41/46; C12M 35/02; C12M 35/04; C12M 23/16; C12M 25/02

(57) ABSTRACT

The present disclosure relates to a piezoelectric membrane microelectrode array for spatially resolved electrical or mechanical stimulation and simultaneous spatially resolved measurement of electrical or mechanical activity of biological material. The array comprises at least two membrane microelectrode units, that are both arranged on a common substrate.

### CROSS REFERENCE TO RELATED APPLICATIONS

This is a continuation application of co-pending international patent application PCT/EP2021/077671, filed Oct. 7, 2021 and designating the United States, which was published in German as WO 2022/078864 A1, and claims priority to German patent application DE 10 2020 126 759.2, filed Oct. 12, 2020, both of which are incorporated herein by reference in their entirety.

### FIELD

The present invention relates to a piezoelectric membrane microelectrode array and a membrane microelectrode unit. The piezoelectric membrane microelectrode array comprises at least two membrane microelectrode units arranged on a substrate. The present invention further relates to a method of manufacturing the membrane microelectrode unit and the piezoelectric membrane microelectrode array. Moreover, the present invention relates to uses of the piezoelectric membrane microelectrode array.

### BACKGROUND

In the field of medical research, cell cultures are an important element. Cells can be studied in vitro under controlled conditions, which is a useful alternative to animal testing. For example, changes within cells can be observed after contact with a test substance or by applying a voltage. Furthermore, mechanical changes of the cells can be measured.

In order to measure electrophysiological properties of cells, for example, the so-called patch clamp technique was developed by Erwin Neher and Bert Sakmann in 1976. Here, the function of individual cellular membrane proteins can be determined. By making it possible to observe the electrical behavior of membrane proteins on individual molecules, the patch-clamp technique revolutionized electrophysiological research.

Over time, this technique was further refined so that a measurement of mechanical activity of the material under investigation could also be detected by means of the patch clamp technique. An important contribution to this was made, for example, by P-C. Zhang, A. M. Keleshian, and F. Sachs, “Voltage-induced membrane movement,” Nature 413, 428 (2001) and T. D. Nguyen, N. Deshmukh, J. M. Nagarah, T. Kramer, P. K. Purohit, M. J. Berry, and M. C. McAlpine, “Piezoelectric nanoribbons for monitoring cellular deformations,” Nature Nanotechnology 7, 587 (2012). Here, for the first time, the patch-clamp technique was combined with an atomic force microscope (AFM) or piezoelectric thin film to produce an electromechanical biosensor. With the help of this biosensor, it is possible to detect the mechanical activity of the cell by applying an electrical voltage with the patch-clamp technique to the cell under investigation using the AFM or the piezoelectric thin film.

A disadvantage of this new technique, however, is the complex experimental setup of the measuring instruments. This results in the additional disadvantage that the measurement is reserved for experienced users only and the individual measurements are very time-consuming due to the effort involved. For this reason, full automation of the measurement is also difficult to realize.

Furthermore, the disadvantage arises that the biological material, preferably electrogenic cells, can only be measured locally. This results from the fact that the measuring electrode must be repositioned for each measurement, which has the additional disadvantage that the cells can be damaged during the measuring process.

Furthermore, the biological material usually has to be prepared in a complex manner, since the outer cell membrane of the cells to be investigated is rarely accessible without restrictions for the patch clamp technique.

WO 2020/064440, on the other hand, describes a platform that enables local stimulation of a biological material by applying an electric field, the platform comprising shear piezoelectric materials.

### SUMMARY

Against this background, it would among other objects be desirable to provide an apparatus which enables improved and/or simpler analysis of biological material. In particular, it would be desirable to overcome one or more of the disadvantages described above. Furthermore, it would be desirable to improve the reproducibility of measurement results.

According to a first aspect of the present disclosure, there is provided a piezoelectric membrane microelectrode array for spatially resolved electrical and/or mechanical stimulation and simultaneous (concurrent) spatially resolved measurement of electrical and/or mechanical activity of biological material, wherein the piezoelectric membrane microelectrode array comprises at least two membrane microelectrode units, the membrane microelectrode units being arranged on a substrate; wherein the or each of the two membrane microelectrode units comprises at least one piezoelectric membrane adapted to mechanically stimulate and/or measure mechanical activity of biological material, wherein the at least one piezoelectric membrane comprises a piezoelectric film, wherein the piezoelectric film is arranged on the substrate, wherein the piezoelectric film is deformable; and wherein the or each of the two membrane microelectrode units comprises at least one first microelectrode adapted to electrically stimulate and/or measure electrical activity of biological material.

According to another aspect of the present disclosure, there is provided a membrane microelectrode unit, wherein the membrane microelectrode unit is arranged on a substrate; wherein the membrane microelectrode unit comprises at least one piezoelectric membrane adapted to mechanically stimulate or measure mechanical activity of biological material, wherein the at least one piezoelectric membrane comprises a piezoelectric film, wherein the piezoelectric film is arranged on the substrate, wherein the piezoelectric film is deformable; and wherein the membrane microelectrode unit comprises at least one first microelectrode adapted to electrically stimulate or measure electrical activity of biological material.

Further, there is provided a multiwell plate for electrical or mechanical stimulation and simultaneous (concurrent) measurement of electrical or mechanical activity of biological material, wherein the multiwell plate comprises at least one receptacle and at least one membrane microelectrode unit, wherein the at least one receptacle forms a receiving space for the biological material and optionally culture medium, and wherein the at least one receptacle comprises a bottom, wherein the at least one membrane microelectrode unit forms the bottom of the receptacle.

According to another aspect of the present disclosure, there is provided a method of manufacturing a membrane microelectrode unit, the method comprising the steps of:
a) providing a substrate;
b) fabricating a first microelectrode, wherein the fabricating comprises the steps of:
   i) applying a first conductive layer; and
   ii) structuring the first microelectrode out of the first conductive layer;
c) depositing a piezoelectric film on the substrate.

Further, a method of manufacturing a piezoelectric membrane microelectrode array is provided, the method comprising the steps of:
A) providing at least two membrane microelectrode units in at least one receptacle, wherein the at least two membrane microelectrode units form the bottom of the receptacle; and optionally.
B) providing at least one counter electrode, wherein the counter electrode is arranged within the receptacle.

Furthermore, the use of a piezoelectric membrane microelectrode array is provided for electrical, mechanical, optical and/or biochemical spatially resolved stimulation of biological material, or for spatially resolved measurement of electrical and/or mechanical activity of biological material triggered by electrical, mechanical, optical and/or biochemical stimulation, mechanical, optical and/or biochemical stimulation, or for spatially resolved measurement of electrical and/or mechanical activity of biological material and for spatially resolved stimulation of the biological material, wherein the measurement and the stimulation are simultaneous (concurrent or at the same time), or as an immunosensor, gas sensor or as a nanogenerator.

In the context of the present disclosure, “simultaneous” means that the biological material can be stimulated and measured at the same time and additionally in a spatially resolved manner, i.e., at different locations, in particular at different locations simultaneously. In the context of the present disclosure, the term “simultaneous” may be used as a synonym for “concurrent” or “at the same time” and vice versa. In other words, the device may be arranged for spatially resolved electrical stimulation and/or spatially resolved mechanical stimulation. The device may be adapted for simultaneous spatially resolved measurement of electrical activity and/or spatially resolved measurement of mechanical activity. The device may be adapted for simultaneous spatially resolved mechanical and/or electrical stimulation and/or spatially resolved measurement of electrical and/or mechanical activity. The device may be adapted for simultaneous or concurrent spatially resolved electrical stimulation and spatially resolved mechanical stimulation. The device can be adapted for simultaneous or concurrent spatially resolved measurement of electrical activity and spatially resolved measurement of mechanical activity. Electrodes for spatially resolved measurement of electrical activity of biological material may additionally be provided with piezoelectric membranes for spatially resolved measurement of mechanical activity.

The proposed piezoelectric membrane microelectrode array (microelectrode array with piezoelectric membrane) provides a simultaneous measurement and stimulation array that enables spatially resolved measurement and simultaneous (concurrent) stimulation of biological material, such that the biological material can be electrically and/or mechanically stimulated and simultaneously measured. This is achieved by the interaction of the at least one piezoelectric membrane and the at least one first microelectrode. Thereby, biological material can be investigated in a spatially resolved manner as to which effects different (spatially resolved) stimulations, e.g. electrical and/or mechanical, have on the material. For example, electrical stimulation and simultaneous (concurrent) mechanical measurement of biological material are possible in a spatially resolved manner, even though this is not possible with the means available in the prior art to date. On the other hand, mechanical stimulation and simultaneous electrical measurement of biological material is also possible in a spatially resolved manner.

In the context of the present disclosure, “biological material” can be tissue, cell composites or individual cells, which are preferably of human or animal origin. Preferably, the biological material can be excited electrically or mechanically, i.e., by applied electrical voltage or by mechanical deformation of the biological material. For example, the biological material can be electrogenic cells such as cardiomyocytes or nerve cells.

Stimulation of the biological material may be achieved electrically or mechanically by the piezoelectric membrane microelectrode array. In the context of the present disclosure, “stimulate” can indicate that the biological material is stimulated, in which case the biological material may, for example, exhibit altered electrical and/or mechanical properties.

The biological material can also be stimulated optically, for example light-induced, whereby the biological material can respond by changing its electrical and/or mechanical properties. Furthermore, it is possible that the biological material is stimulated biochemically; for example, by the addition of active substances to which the biological material responds by changing its electrical and/or mechanical properties. Regardless of the type of stimulation of the biological material, the piezoelectric membrane microelectrode array makes it possible to simultaneously measure both the electrical and the mechanical change or response of the biological material.

The “electrical stimulation” can be achieved via the first microelectrode. For this purpose, the microelectrode is in the immediate vicinity of the biological material or is in contact with the biological material, so that electrical voltage can be delivered/transferred to the biological material by the microelectrode. Since the proposed array is used for stimulation and measurement of biological material, the voltage values are in particular below 3 volts, in particular below 2 volts, in particular below 1.3 volts.

The “mechanical stimulation” can be achieved via the piezoelectric membrane. For this purpose, the piezoelectric membrane is in the immediate vicinity of the biological material or is in contact with the biological material. For mechanical stimulation, the piezoelectric membrane is mechanically deformed by an applied electrical voltage due to the piezoelectric effect, so that the biological material in contact with the piezoelectric membrane is mechanically stimulated, i.e. is itself deformed.

In the context of the present disclosure, “measured electrical activity of biological material” can refer to voltage changes within the biological material, which may be achieved in particular by stimulation, for example mechanical, optical or biochemical.

In the context of the present disclosure, the “measured mechanical activity of biological material” can be a deformation of the biological material, which may be achieved in particular by stimulation, for example electrically, optically or biochemically.

The measurement of the activity of the biological material can be achieved by measuring the electrical voltage, or measuring a voltage change triggered by the biological material and/or by measuring the mechanical activity originating from the activated biological material deforming the piezoelectric membrane. The measurement of the mechanical activity is enabled by the piezoelectric membrane, whereas the electrical activity can be measured by the first microelectrode. Accordingly, the microelectrode can be used simultaneously for electrical measurement and stimulation alongside the piezoelectric membrane.

An advantage of the proposed solution can be that the measurement results are improved by achieving better reproducibility. With the patch clamp technique, the measurement results can strongly depend on the operator. In particular, the electrical stimulation and/or measurement provided from above can vary greatly. In some cases, the cells to be measured are damaged in the process. Furthermore, the relative position of electrical and mechanical stimulation and/or measurement often varies significantly from sample to sample.

The membrane microelectrode unit may comprise at least one piezoelectric membrane. Accordingly, the membrane microelectrode unit may have one, two, three, four, five, or more piezoelectric membranes. With a plurality of membranes, the individual membranes can, for example, each be used independently as a measurement membrane or a stimulation membrane.

The membrane microelectrode unit may comprise at least one first microelectrode. Accordingly, the membrane microelectrode unit may have one, two, three, four, five, or more microelectrodes. With a plurality of microelectrodes, the individual electrodes can, for example, each be used independently as a measurement microelectrode or a stimulation microelectrode.

The piezoelectric film can be deformable. “Deformable” in this context can indicate that the film bends from a rest position, due to an applied voltage or due to the activated biological material deforming the piezoelectric film, to an excited position, wherein the deformation of the piezoelectric film is dependent on the size of the piezoelectric film. For example, for a round piezoelectric film with a diameter of about 200 μm, the differences from the rest position of the center of the film to a deformed/excited position of the center of the film can range from 10 to 2000 nm, in particular from 100 to 1000 nm, in particular from 250 to 500 nm.

Preferably, the proposed piezoelectric membrane microelectrode array can be used for both measurement and stimulation. It is to be understood that a corresponding measurement and/or stimulation arrangement may be connected for this purpose. Optionally, the array may comprise at least one measurement and control unit, the latter being electrically connected to the piezoelectric membrane and/or to the at least one first microelectrode via conductive tracks. According to a preferred embodiment, each individual membrane microelectrode unit can be individually controlled by means of the measurement and control unit, whereby the biological material can be stimulated and measured by each individual membrane microelectrode unit. According to a further preferred embodiment, several, for example two, three, four, five, six, seven or eight membrane microelectrode units can also be controlled by a common measurement and control unit. Alternatively, each individual piezoelectric membrane or microelectrode may be controlled individually, which is a preferred embodiment. Optionally, a measurement amplifier can be arranged on the substrate and connected to the piezoelectric membrane electrode array via conductive tracks on the substrate. This allows even small signal changes to be detected. Furthermore, relatively interference-free measurement of weak signals is possible.

According to a first embodiment of the disclosed piezoelectric membrane microelectrode array, the first microelectrode and the piezoelectric film may be spaced apart.

In the context of the present disclosure, “spaced apart” can indicate that the first microelectrode is arranged spatially separated from the piezoelectric film; i.e., that the microelectrode is not disposed above or below the piezoelectric film within the piezoelectric membrane microelectrode array; but next to each other or side-by-side. However, the spacing can be chosen to be small such that spatially resolved stimulation/measurement can take place. The distance can be chosen large enough such that the first microelectrode and the piezoelectric film do not influence or interfere with each other during stimulation/measurement.

This embodiment has the advantage that the first microelectrode and the piezoelectric film do not interfere with each other during a measurement, i.e. stimulation and simultaneous measurement of the biological material, which may lead to measurement errors. This may occur if the first microelectrode and the piezoelectric film are arranged directly adjacent, i.e. without any separation.

According to an embodiment of the piezoelectric membrane microelectrode array, the substrate can comprise at least two regions, a first region having a first layer thickness and a second region having a second layer thickness, wherein the first layer thickness is greater than the second layer thickness, and wherein the piezoelectric film is disposed within the second region of the substrate.

The substrate can be a silicon-on-insulator (SOI) wafer or a pre-patterned or pre-structured substrate. With this embodiment, the biological material can preferably be mechanically stimulated and/or mechanically measured with the piezoelectric membrane. Alternatively, the substrate can be a printed circuit board (PCB). Further, a PCB can be provided as a holder for a plurality of substrates.

The layer thickness of the first region is, for example, in the range of 200 to 2000 μm, in particular in the range of 200 to 1000 μm, in particular in the range of about 500 μm. The layer thickness of the second region is in particular in the range of 1 to 50 μm, in particular in the range of 1 to 25 μm, in particular in the range of 1 to 10 μm.

According to an embodiment of the piezoelectric membrane microelectrode array, the first microelectrode can be arranged within the first or second region of the substrate.

According to the first alternative, the first microelectrode is arranged within the first region of the substrate, i.e. within the substrate region with the greater film thickness. According to this embodiment, the first microelectrode is spaced apart not only from the piezoelectric film but from the piezoelectric membrane. This embodiment has the advantage that, when the first microelectrode stimulates or measures, the piezoelectric membrane is not disturbed by the first microelectrode and vice versa.

According to the second alternative, the first microelectrode is arranged within the first region of the substrate, i.e. within the substrate region with the smaller layer thickness. According to this embodiment, the first microelectrode is arranged within the piezoelectric membrane. This embodiment offers the advantage of a more compact design, since the distances between the first microelectrode and the piezoelectric film are shorter than the same distances of the first alternative. Furthermore, measurement and stimulation can be performed closer together.

According to an embodiment of the piezoelectric membrane microelectrode array, the piezoelectric membrane comprises at least one first electrode, wherein the at least one first electrode is electrically conductively coupled to the piezoelectric film.

According to this embodiment, the at least one first electrode can preferably be arranged between the substrate and the piezoelectric film. Alternatively or in addition, an electrode can be provided as a top electrode on the piezoelectric film. In this case, an insulating layer can be provided over the top electrode to isolate the top electrode of the piezoelectric film from the biological material or a nutrient solution. Alternatively, the electrode can be in contact with the biological material/in close proximity to the biological material. In this case, contact can be made, for example, via a conductive nutrient medium. For example, the electrode can be made of or can comprise any of the following materials: Au, Pt, TiN or conductive oxides such as SrRuO₃ or SrTiO₃ doped with Nb. Preferably, the first electrode has a layer thickness of 70 to 130 nm, in particular of 80 to 120 nm, in particular of 90 to 110 nm. An electrode layer thickness of about 100 nm is particularly preferred.

According to an embodiment of the disclosed piezoelectric membrane microelectrode array, the first electrode is an interdigital electrode.

In the context of the present disclosure, the term “interdigital electrode” may be used as a synonym for “interdigital transducer” (IDT for short). According to this embodiment, the first electrode is arranged within the piezoelectric membrane on the piezoelectric film, such that the piezoelectric film is at least partially arranged between the substrate and the first electrode. According to this embodiment, the interdigital electrode can preferably have two electrodes, the two electrodes preferably being parallel to each other and bounding the piezoelectric film.

According to an embodiment of the piezoelectric membrane microelectrode array, the piezoelectric film consists of or comprises a ferroelectric material, which is preferably selected from lead-free oxides having a perovskite structure, in particular 0.5(Ba_{0.7}Ca_{0.3})TiO₃-0.5Ba(Zr_{0.2}Ti_{0.8})O₃ or K_{0.5}NaNbO_{0.53}; CMOS compatible ferroelectrics, in particular Al₁₋ₓScₓN with 0.2≤x≤0.5 or HfZrO_{0.50.52}; and ferroelectric polymers, in particular polyvinylidene fluoride or ferroelectrics with multiferroic properties, in particular BiFeO₃.

These materials have been found to be particularly advantageous for mechanical stimulation and mechanical measurement of biological material. Lead-free ferroelectrics are particularly preferred over lead-containing ferroelectrics, such as lead zirconate titanate (PZT), because of the toxicity of lead.

According to an embodiment, the piezoelectric film may have a film thickness of 100 to 3000 nm, in particular of 500 to 1500 nm, in particular a film thickness of about 1000 nm.

According to an embodiment of the piezoelectric membrane microelectrode array, the piezoelectric membrane is spaced apart from the first microelectrode at a distance of 0.5 to 500 μm, in particular at a distance of 0.5 to 50 μm, in particular at a distance of 0.5 to 5 μm.

This embodiment has the advantage that if the biological material is stimulated by the first microelectrode, the piezoelectric membrane is not disturbed by the applied voltage of the first microelectrode, so that mechanical activity can be measured with the piezoelectric membrane without interference. The same applies vice versa, so that the piezoelectric membrane stimulating biological material does not interfere with the first microelectrode during measurement. Accordingly, with this embodiment measurement errors can be reduced.

According to an embodiment of the piezoelectric membrane microelectrode array, the first microelectrode is arranged within the piezoelectric membrane.

According to this embodiment, particularly space-saving/compact membrane microelectrode units can be provided so that a single piezoelectric membrane microelectrode array can have more membrane microelectrode units on the same area of the substrate than an array in which the first microelectrode is not located within the piezoelectric membrane.

According to an embodiment of the piezoelectric membrane microelectrode array, the piezoelectric membrane is configured as a piezoelectric cantilever or a piezoelectric nanoribbon.

In the context of the present disclosure, a “piezoelectric cantilever” may be a particular embodiment of the piezoelectric membrane or diaphragm, wherein the piezoelectric membrane is fixedly clamped in the substrate on one side so that the diaphragm is at least partially freely suspended. Such a piezoelectric cantilever is described by way of example in US 2005/0193823 A1, but not yet in the context of a membrane microelectrode unit.

In the context of the present disclosure, a “piezoelectric nanoribbon” may be a particular embodiment of the piezoelectric membrane, wherein the piezoelectric membrane is tightly constrained in the substrate on two opposite sides such that the membrane is at least partially free hanging. An example of such a piezoelectric nanoribbon is described in the paper T. D. Nguyen et al. “Piezoelectric nanoribbons for monitoring cellular deformations,” Nature Nanotechnology 7, 587 (2012).

According to an embodiment of the piezoelectric membrane microelectrode array, the piezoelectric membrane microelectrode array comprises a receptacle and optionally at least one counter electrode, wherein the receptacle forms a receiving space for the biological material and optionally culture medium, and wherein the receptacle comprises a bottom, wherein the at least one membrane microelectrode unit forms the bottom of the receptacle, and wherein the optional counter electrode can measure electrical signals originating from the biological material.

In the context of the present disclosure, the “receptacle” may be a sample receptacle capable of storing the biological material. Particularly preferred is a cylindrical receptacle that is closed off at the bottom by the proposed piezoelectric membrane microelectrode array. Preferably, the receptacle is configured such that it can store a culture medium for the biological material, wherein the culture medium is preferably liquid.

In the context of the present disclosure, the term “counter electrode” and “reference electrode” may be used as synonyms. The counter electrode may be configured to measure electrical signals or electrically stimulate the biological material between the first electrode and the counter electrode. The measurement is carried out via potential differences of the counter electrode and the first microelectrode. The counter electrode can be immersed in the culture medium, arranged at the edge of the receptacle or integrated in the substrate.

As mentioned at the outset, another aspect of the disclosure relates to a multiwell plate for electrical and/or mechanical stimulation and simultaneous (concurrent) measurement of electrical and/or mechanical activity of biological material.

The “multiwell plate” can be, for example, a microtiter plate. A multiwell plate can comprise a plurality of receptacles for receiving or holding biological material or nutrient solution and biological material, for example, the multiwell plate can comprise 6, 12, 24, 48, 96, or 384 receptacles. One or more of the receptacles can comprise at least one membrane microelectrode unit at the bottom of the respective receptacle. The bottom of a plurality of receptacles may be formed by a common substrate with a plurality of one membrane microelectrode units, wherein a plurality of the receptacles each comprise an associated membrane microelectrode unit. Alternatively, receptacles can comprise separate substrates each comprising at least one membrane microelectrode unit. One advantage is that the yield in manufacturing can be improved. The respective substrates may for example be arranged on a common PCB. A multiwell plate with 24 or 96 receptacles is particularly preferred. In the context of the present disclosure, the term “receptacle” and “cavity” may be used as synonyms.

As mentioned at the outset, another aspect of the disclosure relates to a method of manufacturing a membrane microelectrode unit.

The “depositing of the piezoelectric film” on the substrate in step c) can be achieved by a thin film process. In this step, the piezoelectric film is applied or deposited onto the substrate.

The “applying the first conductive layer” in step b) i) can be achieved by a thin film process. Hereby, an electrically conductive layer is applied or deposited in a preferred layer thickness of approx. 100 nm.

The “structuring the first microelectrode out of the first conductive layer” in step b) ii) can be achieved using optical lithography. In this step, a conductive path and contact pads of the first microelectrode can also be structured out.

The method of manufacturing a membrane microelectrode unit can comprise the step b):
b) fabricating the first electrode and a first microelectrode on the substrate,
wherein the fabricating comprises the following steps:
   i) depositing a first conductive layer onto the substrate; and
   ii) structuring out the first electrode and the first microelectrode from the first conductive layer;
      wherein the piezoelectric film is in step c) deposited onto the first electrode.

According to this embodiment, the first electrode is arranged between the substrate and the piezoelectric film. This embodiment provides the advantage that the first electrode and the first microelectrode can be structured out of the same conductive layer. Since no (metallic) top electrode including conductive path and insulator is necessary on the membrane according to this embodiment, they can also not negatively influence the mechanical properties of the membrane. According to this embodiment, the first electrode is preferably arranged inside the piezoelectric membrane so that the first electrode is spaced apart from the first microelectrode.

According to an embodiment of the method for manufacturing a membrane microelectrode unit, an insulator is at least partially applied onto the first microelectrode and/or the first electrode.

In the context of the present disclosure, the insulator may consist of or comprise, for example, Si₃ Ni₄. The insulator may be applied onto the first microelectrode and/or the first electrode such that conductive paths of the first microelectrode or the first electrode are insulated. Preferably, those areas that are in direct contact with the biological material and are used for stimulation or measurement of the biological material are not insulated.

According to an embodiment of the process for fabricating a membrane microelectrode unit, the substrate may be structured, preferably by a Bosch process.

In the context of the present disclosure, “structuring” can refer to introducing microstructures into the substrate. Preferably, the microstructures are introduced into the side of the substrate facing away from the biological material. This can be achieved, for example, by the Bosch process. In the context of the present disclosure, the term “Bosch process” can be used as a synonym for “reactive ion deep etching”. In the structuring process, a depth structure is preferably introduced within the piezoelectric membrane. The depth structure is preferably introduced such that the remaining layer thickness is in the range of 1 to 50 μm, in particular in the range of 1 to 25 μm, in particular in the range of 1 to 10 μm.

As mentioned at the outset, another aspect of the disclosure relates to a method of manufacturing a piezoelectric membrane microelectrode array.

The counter electrode can be arranged inside the receptacle. In this case, the counter electrode may only be in contact with the electrolyte/culture medium. Alternatively, the counter electrode can be arranged on the receptacle, for example on the edge of the receptacle. Alternatively, the counter electrode may be arranged on or under the piezoelectric film.

As mentioned at the outset, another aspect of the disclosure relates to the use of the proposed piezoelectric membrane microelectrode array. Hereby, the array can be used for electrical, mechanical, optical, and/or biochemical spatially resolved stimulation of biological material.

Furthermore, the piezoelectric membrane microelectrode array can be used for spatially resolved measurement of electrical and/or mechanical activity of biological material triggered by electrical, mechanical, optical and/or biochemical stimulation.

Further, the piezoelectric membrane microelectrode array can be used for spatially resolved measurement of electrical and/or mechanical activity of biological material and spatially resolved stimulation of the biological material, with the measurement and stimulation occurring simultaneously (concurrently).

Furthermore, the piezoelectric membrane microelectrode array can be used as an immunosensor, gas sensor, or nanogenerator.

According to this embodiment, with an immunosensor the formation of antigen-antibody complexes can be measured. This can be detected, for example, by electrical signals as well as by property changes such as mass changes. Furthermore, according to this embodiment, with a gas sensor, the shift of the resonance frequency of the membranes can be measured when gas particles dock there. Furthermore, according to this embodiment, with the nanogenerator vibrations from the environment that cause the piezoelectric membranes to vibrate can be measured as an electrical voltage via the direct piezoelectric effect.

It is to be understood that the above features and those to be explained below can be used not only in the respective shown combination, but also in other combinations or on their own, without departing from the scope of the present invention.

### BRIEF DESCRIPTION OF THE DRAWINGS

Exemplary embodiments of the disclosure are shown in the drawing and are explained in more detail in the following description. The figures show:

FIG. 1 a cross-section of a first embodiment of the membrane microelectrode unit according to an aspect of the present disclosure,

FIG. 2 a cross-section of a second embodiment of the membrane microelectrode unit according to an aspect of the present disclosure,

FIG. 3 a cross-section of a third embodiment of the membrane microelectrode unit according to an aspect of the present disclosure,

FIG. 4A a cross-section of a fourth embodiment of the membrane microelectrode unit according to an aspect of the present disclosure,

FIG. 4B a top view of the fourth embodiment of the membrane microelectrode unit according to an aspect of the present disclosure,

FIG. 4C a top view of a fourth embodiment of the piezoelectric membrane microelectrode array according to an aspect of the present disclosure,

FIG. 5 a cross-section of a fifth embodiment of the membrane microelectrode unit according to an aspect of the present disclosure,

FIG. 6A a cross-section of a sixth embodiment of the membrane microelectrode unit according to an aspect of the present disclosure,

FIG. 6B a cross-section of a seventh embodiment of the membrane microelectrode unit according to an aspect of the present disclosure,

FIG. 7 a cross-section of an eighth embodiment of the membrane microelectrode unit according to an aspect of the present disclosure,

FIG. 8A a top view of a ninth embodiment of the piezoelectric membrane microelectrode array according to an aspect of the present disclosure, and

FIG. 8B a top view of a tenth embodiment of the piezoelectric membrane microelectrode array according to an aspect of the present disclosure.

### DESCRIPTION OF THE PREFERRED EMBODIMENTS

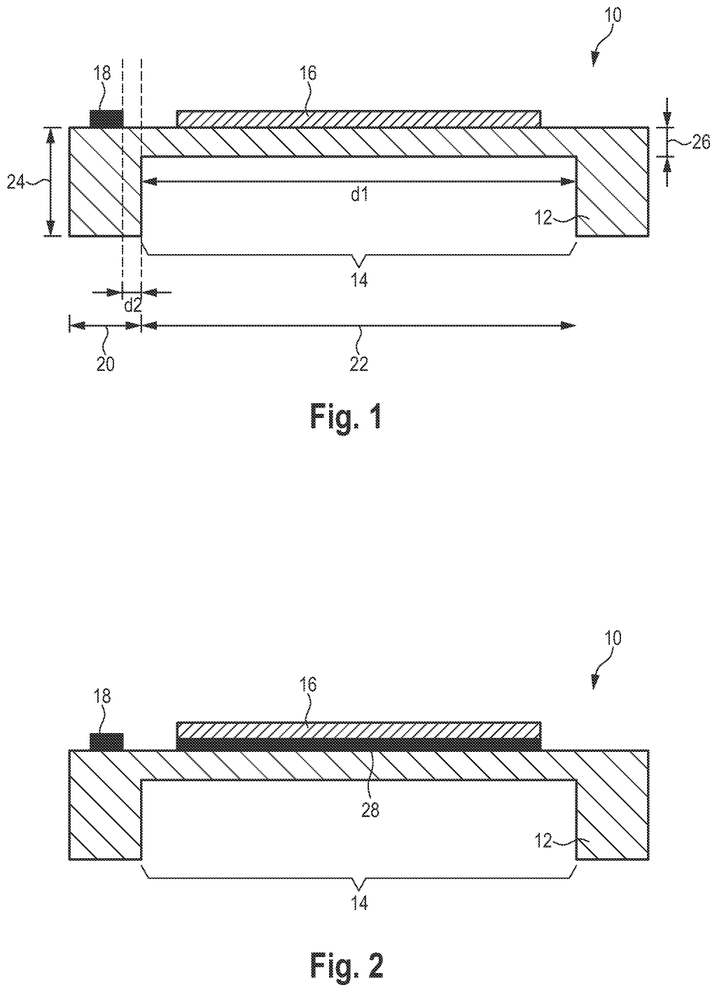

FIG. 1 shows a cross-sectional view of a first embodiment of the proposed membrane microelectrode unit 10, wherein the membrane microelectrode unit 10 is arranged on a substrate 12. The membrane microelectrode unit 10 comprises a piezoelectric membrane 14 having a diameter d1, wherein the piezoelectric membrane 14 comprises a piezoelectric film 16, wherein the piezoelectric film 16 is arranged on the substrate 12, and wherein the piezoelectric film 16 is deformable. The piezoelectric membrane 14 can be configured to be adapted for both mechanical stimulation and measurement of mechanical activity of biological material.

Spaced apart from the piezoelectric film 16, the membrane microelectrode unit 10 includes at least a first microelectrode 18. The microelectrode 18 is arranged on the substrate 12. The distance of the microelectrode 18 from the piezoelectric membrane 14 is indicated by d2. Preferably, the distance d2 is 0.5 to 500 μm. Alternatively, the distance d2 may be 0. The microelectrode 18 can be configured such that it is adapted for both electrical stimulation and measurement of electrical activity of biological material.

According to this embodiment, the substrate 12 comprises two regions 20, 22; a first region 20 having a first film thickness 24 and a second region 22 having a second film thickness 26, wherein the first film thickness 24 is greater than the second film thickness 26. As can be seen in FIG. 1, the piezoelectric film 16 is arranged within the second region 22 of the substrate 12. According to an aspect of the present disclosure the invention, the substrate 12 can be a pre-structured substrate or a silicon-on-insulator wafer, which can be (backside) structured, for example, during the manufacturing process using the Bosch process or another structuring process, such that the substrate 12 comprises the respective regions 20 and 22.

As can be seen in FIG. 1, the piezoelectric film 16 extends only partially across the piezoelectric membrane 14. In an embodiment not shown, the piezoelectric film 16 may extend across the entire piezoelectric membrane 14.

The piezoelectric film 16 can be consist of or comprise a ferroelectric material. This material is particularly advantageous for mechanical stimulation and mechanical measurement of biological material. According to an aspect of the present disclosure, those ferroelectric materials are preferred which do not cause toxicity to the biological material. Accordingly, lead-containing ferroelectrics such as lead zirconate titanate (PZT) are less preferred.

FIG. 2 shows a cross-sectional view of a second embodiment of the membrane microelectrode unit 10 according to an aspect of the present disclosure. It differs from the unit 10 shown in FIG. 1 just by an additional first electrode 28, which is arranged within the piezoelectric membrane 14. The first electrode 28 is electrically conductively coupled to the piezoelectric film 16.

In the shown embodiment, the first electrode 28 is arranged between the piezoelectric film 16 and the substrate 12. Accordingly, the first electrode 28 is not in direct contact with the biological material. According to an aspect of the present disclosure, the first electrode 28 is electrically conductive, so that it also consists of or comprises a conductive material. Preferably, the electrode layer thickness of the first electrode 28 is about 100 nm.

According to an aspect of the present disclosure, the first microelectrode 18 and the first electrode 28 can be connected to one or more measurement and control units via conductive paths (not shown). An electrically conductive culture medium/electrolyte solution can for example serve as the counter electrode. Alternatively, a dedicated counter electrode can be provided to provide a closed circuit. For example, the counter electrode immersed in the electrolyte solution can consist of or comprise AgCl.

As an alternative to the embodiment shown in FIG. 2, the first electrode 28 can also be arranged above the piezoelectric film 16 (see FIG. 6).

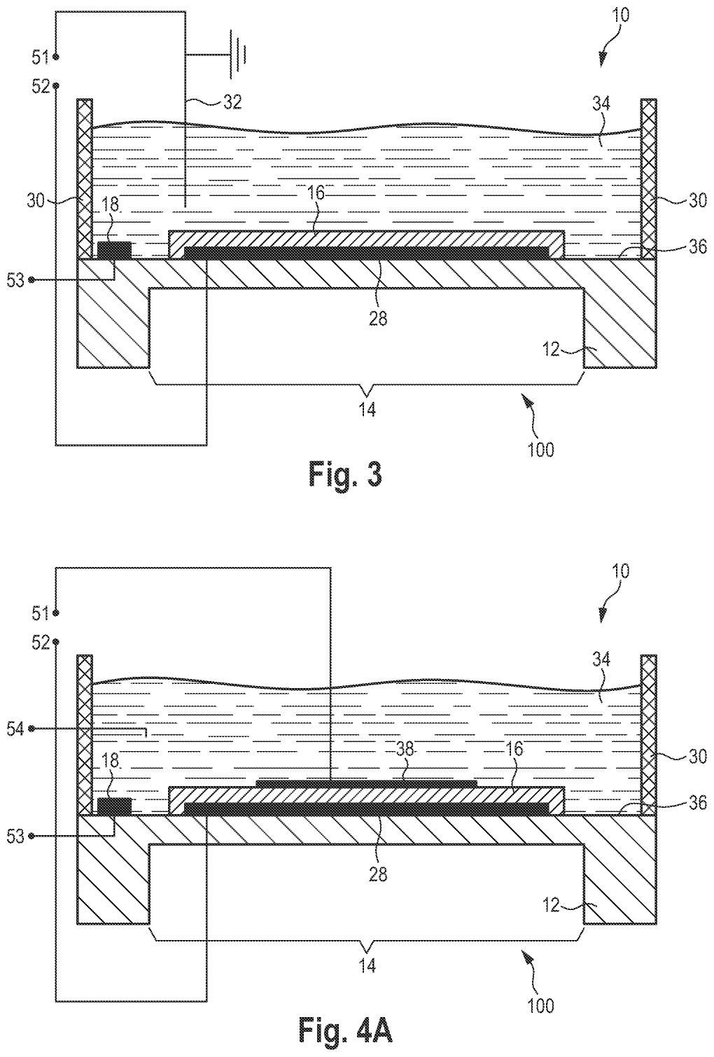

FIG. 3 shows a cross-sectional view of a third embodiment of the membrane microelectrode unit 10 according to an aspect of the present disclosure. The third embodiment differs from the second embodiment in that it includes an additional receptacle 30 and an additional counter electrode 32. Here, the receptacle 30 forms a receiving space for the biological material and the culture medium/electrolyte solution 34. As shown in FIG. 3, the receptacle 30 has a bottom 36, wherein the membrane microelectrode unit 10 forms the bottom 36 of the receptacle 30.

The counter electrode 32 or reference electrode 32 can be adapted such that it serves, for example, as a ground connection. In this case, the measurement is performed via potential differences of the counter electrode 32 and the first microelectrode 18 and via potential differences of the counter electrode 32 and the first electrode 28. In this embodiment, the counter electrode 32 is immersed in the culture medium or the electrolyte 34. According to an aspect of the present disclosure, the counter electrode 32 can also be arranged on the receptacle 30 or in the membrane microelectrode unit 10.

In FIG. 3, the receptacle 30 is configured as a cylindrical sample receptacle, whereby this is delimited at the bottom by the piezoelectric membrane microelectrode unit 10 according to an aspect of the present disclosure. Other configurations, such as a funnel-shaped sample receptacle, are also possible.

For connection to a measuring and/or stimulation device connections 51, 52, 53 can be provided. A common ground connection 51 can optionally be provided. For electrical stimulation, an electrical stimulation signal can be provided via connection 53. For measuring a mechanical response to the stimulation, a measurement amplifier, such as a differential amplifier or operational amplifier, can for example be connected to the connections 51, 52. Optionally, the measurement amplifier can be co-integrated on the substrate. This can reduce interference during further processing of the signals, since already amplified signals are routed away from the substrate.

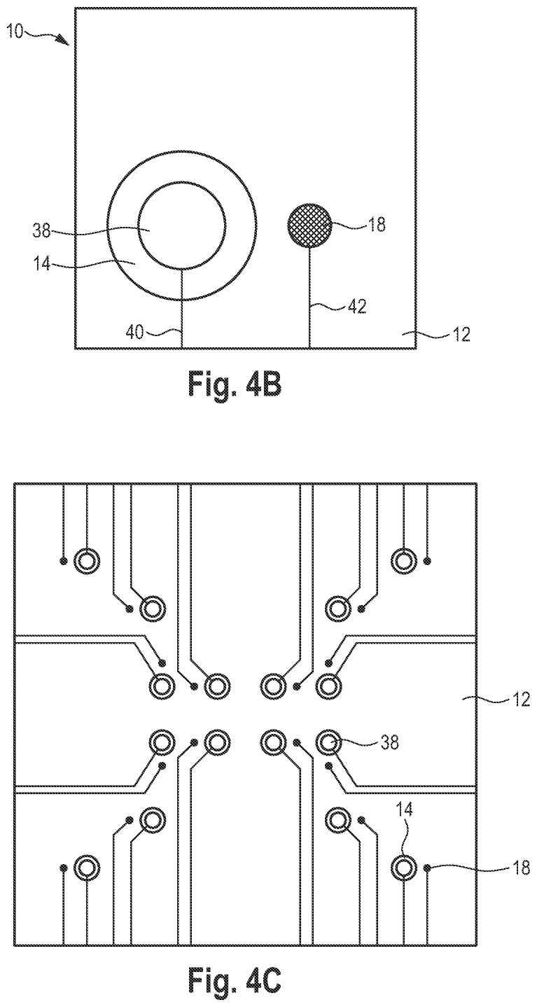

FIG. 4A shows a cross-sectional view of a fourth embodiment of the membrane microelectrode unit 10 according to an aspect of the present disclosure. This embodiment differs from the third embodiment in that a film electrode 38 is arranged above the piezoelectric film 16. In the shown embodiment, the film electrode 38 is smaller in diameter than the piezoelectric film 16 and accordingly only partially covers it. Accordingly, in the shown embodiment, the piezoelectric film 16 is at least partially and the film electrode 38 is completely in direct contact with the biological material in a measurement/stimulation. Whereby “direct contact” in this context means that the piezoelectric film 16 and the film electrode 38 may optionally also comprise an additional insulator layer, i.e., an insulator may be present between the piezoelectric film 16 or the film electrode 38 and the biological material (not shown). In this embodiment, the counter electrode 54 can be provided via the culture medium or electrolyte 34, for example by immersing a counter electrode 54 in the culture medium or electrolyte 34 (similar to FIG. 3). Alternatively, the film electrode 38 can serve as a counter electrode, e.g., a ground. In this case, no insulator is provided with respect to the biological material. An advantage is a simpler structure, since a common electrode can be provided for electrical and mechanical interaction.

For this fourth embodiment example shown in FIG. 4A, the manufacturing process of the membrane microelectrode unit according to an aspect of the present disclosure will be described here by way of an example. It is to be understood that the individual process steps also apply accordingly to the other embodiments of the other figures and generally for the invention, without departing from the scope of the present invention.

The method for fabricating a membrane microelectrode unit 10 according to an aspect of the present disclosure can be carried out using standard silicon and thin film technology processes. In a first step of the method, the substrate 12 is provided. The substrate 12 is preferably a planar substrate which is, for example, a silicon-on-insulator (SOI) wafer or a pre-structured substrate.

In a subsequent step, a first electrode 28 can be fabricated. Hereby, a conductive layer that will form the first electrode 28 is first applied to the substrate 12, and in a next step, the first electrode 28 can be structured out. For example, the first electrode 28 con consist of or comprise any of the following materials: Pt, TiN, SrRuO₃. In this regard, the first electrode 28 can optionally be deposited with an adhesion promotion layer, for example of Ti or Ta, and/or a buffer layer, for example of SiO2. The adhesion promotion layer and buffer layer are not shown in FIG. 4A. Preferred layer thicknesses here are about 300 nm for the buffer layer, about 10 nm for the adhesion promotion layer and about 100 nm for the first electrode 28.

In a subsequent step, the piezoelectric film 16 is applied onto the substrate 12, or in this embodiment, to the first electrode 28. For example, the piezoelectric film 16 can be grown on the respective top/last layer by a thin film process. Preferably, a piezoelectric film 16 with a layer thickness of 500 to 1000 nm is used.

Subsequently, in a subsequent step, also in a thin-film process, a further conductive layer can be applied onto the substrate 12, or in this embodiment onto the piezoelectric film 16. A layer thickness of about 100 nm is preferred. The conductive layer preferably consists of or comprises the following materials: Au, Pt, TiN or conductive oxides such as SrRuO₃ or SrRuO₃ doped with Nb. In order to be able to form the film electrode 38, it is structured out of the conductive layer in a subsequent step. In addition, corresponding conductive tracks and, if necessary, contact pads can be simultaneously structured out of the conductive layer, for example by optical lithography. The conductive tracks serve a connection between the film electrode 38 and a measurement/control unit (not shown). The manufacturing process of the piezoelectric membrane is based on processes for manufacturing SOI wafers. Such processes are exemplified in M. D. Nguyen et. al, “Optimized electrode coverage of membrane actuators based on epitaxial PZT thin films,” Smart Mater. Struct. 22, 085013 (2013) or in C. T. Q. Nguyen et. al. “Process dependence of the piezoelectric response of membrane actuators based on Pb(Zr_{0.45} Ti_{0.55})O3 thin films,” Thin Solid Films 556, 509 (2014).

In a next step, an insulator, for example of Si₃N₄, can be applied onto the piezoelectric membrane 14, preferably structured in such a way that the conductive tracks and the electrodes 28 and 38 on the piezoelectric membrane 14 are insulated.

For producing the first microelectrode 18, a conductive layer is again applied onto the substrate 12; preferably with a layer thickness of approx. 100 nm. In a subsequent step, the first microelectrode 18 is structured out of this conductive layer; preferably, the associated conductive track and the corresponding contact pads are also structured out in this step. According to an aspect of the present disclosure, the first microelectrode 18 is manufactured in such a way that it is always provided at a distance from the piezoelectric film 16. This has the advantage that the first microelectrode 18 and the piezoelectric membrane 14 do not interfere with each other during measurement or stimulation. In a next step, an insulator can again be applied and structured such that the associated conductive path and the corresponding contact pads are insulated. The structuring out can be performed, for example, by reactive ion etching.

In a further step, the piezoelectric film 16 can, for example wet chemically, be structured in the piezoelectric membrane 14. This allows the ratio of piezoelectric film area to piezoelectric membrane area to be optimized.

If the substrate 12 is a non-pre-structured substrate, the backside—the side of the substrate 12 not facing the biological material—can be structured in a subsequent step so that the areas 22 in which the piezoelectric membrane 14 is arranged have a smaller layer thickness 26. This can be achieved, for example, by Bosch process methods. In a further step, the conductive tracks that are not yet insulated can be insulated.

Lastly, a receptacle 30 may be arranged around the membrane electrode unit 10 such that the membrane electrode unit 10 forms the bottom 36 of the receptacle 30. In case that more than one membrane electrode unit 10 are arranged within the receptacle, a piezoelectric membrane microelectrode array 100 according to an aspect of the present disclosure can be obtained.

FIG. 4B shows a top view of the fourth embodiment of the membrane microelectrode unit 10 according to an aspect of the present disclosure. For the sake of clarity, the receptacle and the counter electrode are not shown in FIG. 4B, the embodiment otherwise corresponding to the embodiment in FIG. 4A.

According to the embodiment example in FIG. 4B, the microelectrode 18, the film electrode 38 and the piezoelectric membrane 14 have a circular shape; according to an aspect of the present disclosure, the electrodes 18 and 38 and membrane 14 can also have other shapes. Furthermore, conductive tracks 40, 42 are shown, which lead to the film electrode 38 on the one hand and to the microelectrode 18 on the other hand. The conductive tracks may be covered with an insulating layer. Alternatively, the conductive tracks can be guided on the back of the substrate, i.e. on the side of the substrate facing away from the biological material. In this way, there is no undesired electrical contacting of the biological material by the conductive track.

In FIG. 4B, the microelectrode 18 is arranged at a distance from the piezoelectric membrane 14. According to an aspect of the present disclosure, the distance between the microelectrode 18 and the piezoelectric membrane 14 is preferably selected to be as small as possible, with this distance preferably corresponding at most to the diameter of the biological material (for example biological cells) cultivated thereon. The advantage of this embodiment is that the microelectrode 18 and the piezoelectric membrane 14 can operate independently of each other. For example, if the microelectrode 18 were placed on top of the membrane 14 instead of next to it, this may affect the mechanical properties of the membrane 14.

According to an aspect of the present disclosure, a piezoelectric membrane microelectrode array 100 comprises at least two membrane microelectrode units 10, but a piezoelectric membrane microelectrode array 100 comprising more than two membrane microelectrode units 10 is preferred. Such an array is shown in FIG. 4C, wherein the individual membrane microelectrode units 10 may correspond to the units of FIG. 4B. In this embodiment, the piezoelectric membrane microelectrode array 100 has sixteen individual membrane microelectrode units 10. In particular, these are configured to be individually controllable. Thus, a plurality of corresponding terminals can be led out of the array, which can be controlled accordingly by a measuring and/or stimulation device. According to an aspect of the present disclosure it is also possible that, for example, multiple membrane microelectrode units 10, for example four, can also be respectively controlled together. Any other number of units 10 that can be controlled together is also possible.

According to an aspect of the present disclosure, the piezoelectric membrane microelectrode array 100 can be used for spatially resolved measurement of electrical and/or mechanical activity of biological material triggered by electrical, mechanical, optical and/or biochemical stimulation. Further, according to an aspect of the present disclosure, the piezoelectric membrane microelectrode array 100 can be used for electrical, mechanical, optical, and/or biochemical spatially resolved stimulation of biological material. Further, according to an aspect of the present disclosure, the piezoelectric membrane microelectrode array 100 can be used for spatially resolved measurement of electrical and/or mechanical activity of biological material and for spatially resolved stimulation of the biological material, wherein the measurement and the stimulation occur simultaneously (concurrently).

If the piezoelectric membranes 10 are deformed by a mechanical tension, an electrical voltage is generated due to the direct piezoelectric effect, which can be recorded, for example, by a (multi-channel) measuring amplifier (not shown). Conversely, the piezoelectric membranes 10 are mechanically deformed by an applied electrical voltage and can thus be used for mechanical stimulation. The microelectrodes 18 adjacent to the piezoelectric membranes 10 can be used simultaneously (concurrently or at the same time) for electrical recording and stimulation.

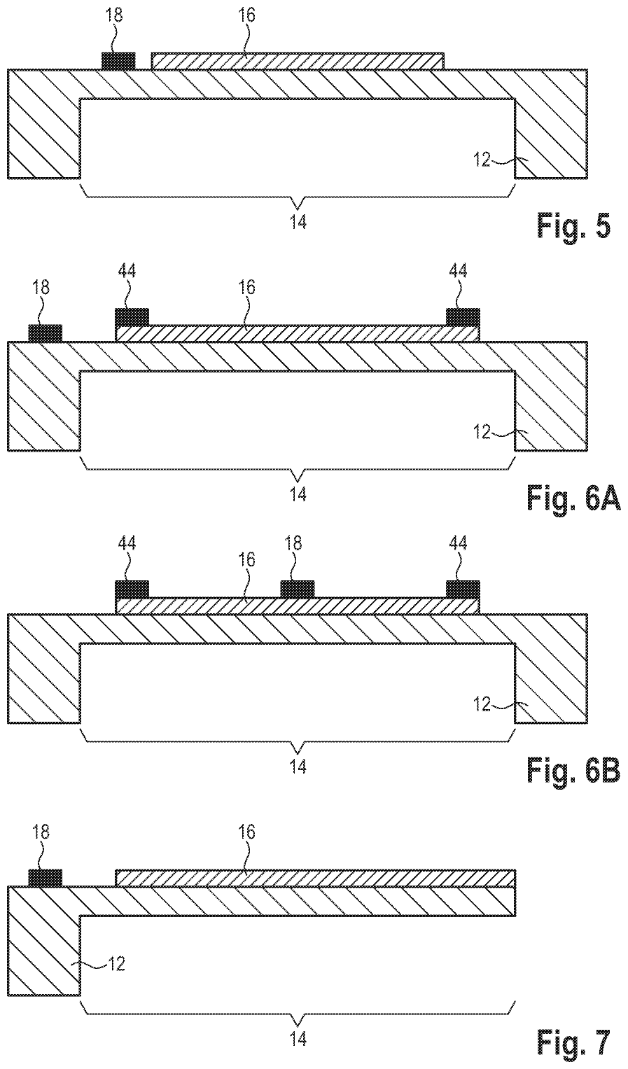

FIG. 5 shows a cross-section of a fifth embodiment of the membrane microelectrode unit 10 according to an aspect of the present disclosure, this embodiment corresponding to the first embodiment except for the difference that the microelectrode 18 is arranged within the piezoelectric membrane 14 on the substrate 12. According to this embodiment, particularly space-saving/compact membrane microelectrode units 10 can be manufactured, such that a single piezoelectric membrane microelectrode array 100 according to an aspect of the present disclosure can have more membrane microelectrode units 10 than an array 100 in which the first microelectrode 10 is not arranged within the piezoelectric membrane 14.

FIG. 6A shows a cross-section of a sixth embodiment of the membrane microelectrode unit 10 according to an aspect of the present disclosure, wherein this embodiment corresponds to the first embodiment except for the difference that the membrane microelectrode unit 10 has an interdigital electrode 44. Here, the interdigital electrode 44 is deposited on the piezoelectric film, and in this embodiment, there are two interdigital electrodes 44 spaced apart from each other. In this embodiment, the microelectrode 18 is present spaced apart from the piezoelectric membrane 14.

FIG. 6B shows a cross-section of a seventh embodiment of the membrane microelectrode unit 10 according to an aspect of the present disclosure, wherein this embodiment corresponds to the sixth embodiment except for the difference that the microelectrode 18 is arranged within the piezoelectric membrane 14, namely on the piezoelectric film 16, in particular centrally on the piezoelectric film 16.

FIG. 7 shows a cross-section of an eighth embodiment of the membrane microelectrode unit 10 according to the invention, this embodiment corresponding to the first embodiment except for the difference that the piezoelectric membrane 14 is configured as a cantilever.

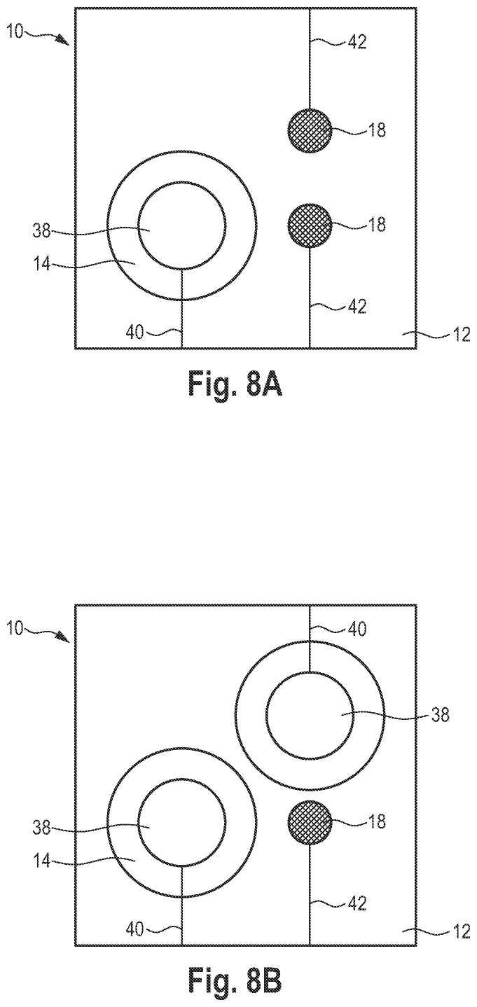

FIG. 8A shows a top view of a ninth embodiment of the membrane microelectrode unit 10 according to an aspect of the present disclosure. This embodiment corresponds to the embodiment shown in FIG. 4B, except that the membrane microelectrode unit 10 has two microelectrodes 18 spaced apart from the piezoelectric membrane 14. In an embodiment not shown, the membrane microelectrode unit 10 according to an aspect of the present disclosure may also comprise more than two, for example three, four, five or more microelectrodes 18. Optionally, the microelectrodes may be arranged symmetrically about the membrane 14. For example, one on the right and one on the left. Multiple microelectrodes 18 may be arranged on a circle around one or more membranes 14.

FIG. 8B shows a top view of a tenth embodiment of the membrane microelectrode unit 10 according to an aspect of the present disclosure. This embodiment corresponds to the embodiment shown in FIG. 4B, except that the membrane microelectrode unit 10 comprises two piezoelectric membranes 14 spaced apart from the first microelectrode 18. In an embodiment not shown, the membrane microelectrode unit 10 according to an aspect of the present disclosure may also comprise more than two, for example three, four, five or more piezoelectric membranes 14. Optionally, the membranes 14 may be arranged symmetrically about the microelectrode 18. For example, one on the right and one on the left. Multiple membranes 14 may be arranged on a circle around one or more microelectrodes 18.

1. A piezoelectric membrane microelectrode array configured to spatially resolved electrical or mechanical stimulation and simultaneous spatially resolved measurement of electrical or mechanical activity of biological material,
wherein the piezoelectric membrane microelectrode array comprises:
at least two membrane microelectrode units, the membrane microelectrode units being arranged on a substrate;
wherein the membrane microelectrode unit comprises at least one piezoelectric membrane adapted to mechanically stimulate or measure mechanical activity of biological material, wherein the at least one piezoelectric membrane comprises a piezoelectric film, the piezoelectric film being arranged on the substrate, wherein the piezoelectric film is deformable; and
wherein the membrane microelectrode unit comprises at least a first microelectrode adapted to electrically stimulate or measure electrical activity of biological material.

2. The piezoelectric membrane microelectrode array according to claim 1, wherein the first microelectrode and the piezoelectric film are spaced apart.

3. The piezoelectric membrane microelectrode array according to claim 1, wherein the substrate comprises at least two regions, a first region having a first layer thickness and a second region having a second layer thickness, wherein the first layer thickness is greater than the second layer thickness, and wherein the piezoelectric film is disposed within the second region of the substrate.

4. The piezoelectric membrane microelectrode array according to claim 3, wherein the first microelectrode is arranged within the first or second region of the substrate.

5. The piezoelectric membrane microelectrode array according to claim 1, wherein the piezoelectric membrane comprises at least one first electrode, wherein the at least one first electrode is electrically conductively coupled to the piezoelectric film.

6. The piezoelectric membrane microelectrode array according to claim 5, wherein the first electrode is an interdigital electrode.

7. The piezoelectric membrane microelectrode array according to claim 1, wherein the piezoelectric film comprises a ferroelectric material.

8. The piezoelectric membrane microelectrode array according to claim 1, wherein the piezoelectric membrane is spaced apart from the first microelectrode at a distance (d2) of 0.5 to 500 μm, 0.5 to 50 μm, or 0.5 to 5 μm.

9. The piezoelectric membrane microelectrode array according to claim 1, wherein the first microelectrode is arranged within the piezoelectric membrane.

10. The piezoelectric membrane microelectrode array according to claim 1, wherein the piezoelectric membrane is configured as a piezoelectric cantilever or a piezoelectric nanoribbon.

11. The piezoelectric membrane microelectrode array according to claim 1, wherein the piezoelectric membrane microelectrode array comprises a receptacle and at least one counter electrode, wherein the receptacle forms a receiving space for the biological material and culture medium, and wherein the receptacle has a bottom, wherein the at least one membrane microelectrode unit forms the bottom of the receptacle, and wherein the counter electrode is adapted to detect electrical signals originating from the biological material.

12. A membrane microelectrode unit configured for electrical or mechanical stimulation and simultaneous measurement of electrical or mechanical activity of biological material, wherein the membrane microelectrode unit is arranged on a substrate;
wherein the membrane microelectrode unit comprises:
at least one piezoelectric membrane adapted to mechanically stimulate or measure mechanical activity of biological material, the at least one piezoelectric membrane comprising a piezoelectric film, wherein the piezoelectric film is arranged on the substrate, wherein the piezoelectric film is deformable; and
wherein the membrane microelectrode unit comprises at least a first microelectrode adapted to electrically stimulate or measure electrical activity of biological material.

13. A multiwell plate configured for electrical and/or mechanical stimulation and simultaneous measurement of electrical or mechanical activity of biological material, said multiwell plate comprising:
at least one receptacle and at least one membrane microelectrode unit according to claim 12, wherein said at least one receptacle forms a receiving space for said biological material and optionally culture medium, and wherein said at least one receptacle comprises a bottom, wherein said at least one membrane microelectrode unit forms the bottom of said receptacle.

14. A method of manufacturing a membrane microelectrode unit comprising:
a) providing a substrate;
b) fabricating a first microelectrode, the fabricating comprises:
i) applying a first conductive layer; and
ii) structuring the first microelectrode out of the first conductive layer; and
c) depositing a piezoelectric film onto the substrate.

15. The method of manufacturing a membrane microelectrode unit according to claim 14, wherein step b) further comprises:
b) fabricating the first electrode and a first microelectrode on the substrate, wherein the fabricating comprises:
i) depositing the first conductive layer onto the substrate; and
ii) structuring the first electrode and the first microelectrode out of the first conductive layer;
wherein the piezoelectric film in step c) is deposited onto the first electrode.

16. The method of manufacturing a membrane microelectrode unit according to claim 14, wherein an insulator is at least partially applied onto one or more of the first microelectrode and the first electrode.

17. The method of manufacturing a membrane microelectrode unit according to claim 14, wherein the substrate is structured by a Bosch-process.

18. A method of manufacturing a piezoelectric membrane microelectrode array wherein the piezoelectric membrane microelectrode array comprises:
at least two membrane microelectrode units, the membrane microelectrode units being arranged on a substrate;
wherein the membrane microelectrode unit comprises at least one piezoelectric membrane adapted to mechanically stimulate or measure mechanical activity of biological material, wherein the at least one piezoelectric membrane comprises a piezoelectric film, the piezoelectric film being arranged on the substrate, wherein the piezoelectric film is deformable; and
wherein the membrane microelectrode unit comprises at least a first microelectrode adapted to electrically stimulate or measure electrical activity of biological material, the method comprising:
A) providing the at least two membrane microelectrode units in at least one receptacle, wherein the at least two membrane microelectrode units form the bottom of the receptacle; and
B) providing at least one counter electrode, wherein the counter electrode is arranged within the receptacle.

19. The piezoelectric membrane microelectrode array according to claim 1, wherein the array is configured for electrical, mechanical, optical and/or biochemical spatially resolved stimulation of biological material.

20. The piezoelectric membrane microelectrode array according to claim 1, wherein the array is configured for spatially resolved measurement of electrical and/or mechanical activity of biological material triggered by electrical, mechanical, optical and/or biochemical stimulation.

21. The piezoelectric membrane microelectrode array according to claim 1, wherein the array is configured for spatially resolved measurement of electrical and/or mechanical activity of biological material and for spatially resolved stimulation of the biological material, wherein the measurement and the stimulation are simultaneous and spatially resolved.

22. The piezoelectric membrane microelectrode array according to claim 1, wherein the array is configured as an immunosensor, a gas sensor, or a nanogenerator.

23. The piezoelectric membrane microelectrode array according to claim 7, which is selected from lead-free oxides having a perovskite structure; CMOS-compatible ferroelectrics; and ferroelectric polymers or ferroelectrics with multiferroic properties.

24. The piezoelectric membrane microelectrode array according to claim 7, which is selected from 0.5(Ba0.7Ca0.3)TiO3-0.5Ba(Zr0.2 Ti0.8)O3; K0.5Na NbO0.53; Al1-xScxN with 0.2≤x≤0.5 or HfZrO0.50.52; polyvinylidene fluoride or BiFeO3.
